(19) United States
(12) Patent Application Publication
(10) Pub. No.: US 2024/0050248 A1
(43) Pub. Date: Feb. 15, 2024

(54) WIRE MANAGEMENT FOR DEVICE DELIVERY SYSTEM

(71) Applicant: W. L. Gore & Associates, Inc., Newark, DE (US)

(72) Inventors: Michael R. Broyles, Flagstaff, AZ (US); Brandon C. Short, Flagstaff, AZ (US)

(21) Appl. No.: 18/267,930

(22) PCT Filed: Dec. 15, 2021
(86) PCT No.: PCT/US2021/063542

§ 371 Date: Jun. 16, 2023

Related U.S. Application Data
(60) Provisional application No. 63/126,982, filed on Dec. 17, 2020.

(51) Int. Cl. A61F 2/954; A61M 25/09

(52) U.S. Cl. CPC A61F 2/954; A61M 25/09041

(57) ABSTRACT

A medical device delivery system comprises an elongate member having a main lumen configured to contain a main guidewire and at least one secondary lumen configured to releasably contain a secondary guidewire, the secondary lumen configured to release a first portion of the secondary guidewire when an implantable medical device is advanced along the main guidewire to a first longitudinal position proximate the first portion of the guidewire and configured to release a second portion of the guidewire when the implantable medical device is advanced along the main guidewire to a second longitudinal position proximate the second portion of the guidewire.

### CROSS-REFERENCE TO RELATED APPLICATION

This application is a national phase application of PCT Application No. PCT/US2021/063542, internationally filed on Dec. 15, 2021, which claims the benefit of Provisional Application No. 63/126,982, filed Dec. 17, 2020, which are incorporated herein by reference in their entireties for all purposes.

### FIELD

The present disclosure relates generally to apparatuses, systems, and methods for managing a plurality of guidewires within a lumen. More specifically, the disclosure relates to apparatuses, systems, and methods for managing a plurality of guidewires prior to and during delivery of a device with branches or usable with other devices or components to be positioned within branches of a lumen.

### BACKGROUND

Treatment of diseased or damaged luminal tissue such as endovascular repair is common. For example, aneurysms occur in blood vessels at sites where, due to age, disease or genetic predisposition of the patient, the strength or resilience of the vessel wall is insufficient to prevent ballooning or stretching of the wall as blood passes through. If the aneurysm is left untreated, the blood vessel wall may expand and rupture, often resulting in death.

Various treatments are available for diseased or damaged luminal tissue. For example, to prevent rupturing of an aneurysm, a stent graft may be introduced into a blood vessel percutaneously and deployed to span the aneurysmal sac. Stent grafts include a graft fabric secured to a cylindrical scaffolding or framework of one or more stents. The stent(s) provide rigidity and structure to hold the graft open in a tubular configuration as well as the outward radial force needed to create a seal between the graft and a healthy portion of the vessel wall and provide migration resistance. Blood flowing through the vessel can be channeled through the luminal surface of the stent graft to reduce, if not eliminate, the stress on the vessel wall at the location of the aneurysmal sac. Stent grafts may reduce the risk of rupture of the blood vessel wall at the aneurysmal site and allow blood to flow through the vessel without interruption.

However, various luminal treatment or repair procedures may occur at a branched site of the lumen. For example, exclusion of an aneurysm at or near a branched artery requires a stent graft to be implanted adjacent to a vascular bifurcation. Often the aneurysm extends into the branch requiring the stent graft to be placed into the branch. A branched stent graft may be required in these cases. Modular stent grafts, having a separate main body and branch component are often preferred in these procedures due to the ease and accuracy of deployment. See U.S. Patent Application No. 2008/0114446 to Hartley et al. for an example of a modular stent graft having separate main body and branch stent components.

In order to properly treat or repair a branched lumen, access to each of the branches may be necessary. Guidewires and catheters are implemented to gain access to the treatment site, where multiple guidewires and catheters may be implemented to gain access to each respective portion of the lumen, including the branches.

### SUMMARY

A wire management device for maintaining guidewires when implanting a device implementing a plurality of wires.

According to one example (“Example 1”), a medical device delivery system comprises an elongate member having a main lumen configured to contain a main guidewire and at least one secondary lumen configured to releasably contain a secondary guidewire, the secondary lumen configured to release a first portion of the secondary guidewire when an implantable medical device is advanced along the main guidewire to a first longitudinal position proximate the first portion of the guidewire and configured to release a second portion of the guidewire when the implantable medical device is advanced along the main guidewire to a second longitudinal position proximate the second portion of the guidewire.

According to another example (“Example 2”), further to Example 1, the elongate member comprises a radially stiff material.

According to another example (“Example 3”), further to Example 1 or 2, the secondary lumen is formed in a wall of the elongate member.

According to another example (“Example 4”), further to any one of Examples 1-3, an opening into the secondary lumen is defined in the elongate member between the secondary lumen and the main lumen along a longitudinal length of the elongate member.

According to another example (“Example 5”), further to any one of Examples 1-3, an opening into the secondary lumen is defined through an outer radial surface of the elongate member along a longitudinal length of the elongate member.

According to another example (“Example 6”), further to Example 1 or 2, the medical device delivery system further comprises a partition positioned in the main lumen of the elongate member, the partition defining the secondary lumen.

According to another example (“Example 7”), further to Example 6, the partition comprises a single sheet of material.

According to another example (“Example 8”), further to any one of Examples 1-7, the secondary lumen is operable to restrict the secondary guidewire from interweaving with the main guidewire along a length of the secondary wire contained by the secondary lumen.

According to one example (“Example 9”), a medical device delivery system comprises an elongate member having a main lumen configured to contain a main guidewire; and a partition removably positioned within the elongate member, the partition forming at least one secondary lumen within the main lumen, the partition configured to contain a secondary guidewire along at least a portion of a longitudinal length of the elongate member, the partition configured to sequentially disrupt along the longitudinal length of the elongate member to free the secondary guidewire from the secondary lumen when the elongate member is removed from surrounding the partition and as an implantable medical device is advanced along the main guidewire, the partition including a disruptable seam.

According to another example (“Example 10”), further to Example 9, a portion of the partition is configured to release a portion of the secondary guidewire into a lumen of the medical device when the portion of the partition is disrupted.

According to another example (“Example 11”), further to Example 9 or 10, the partition subdivides the main lumen to include at least two secondary lumens.

According to another example (“Example 12”), further to any one of Examples 9-11, the partition comprises a sheet of material.

According to another example (“Example 13”), further to any one of Examples 9-12, the partition is folded to form the secondary lumen.

According to another example (“Example 14”), further to Examples 9-13, the partition includes an adhesive for releasably coupling a first edge of the partition to a second edge of the partition.

According to an example (“Example 15”), a medical device delivery system comprises an elongate member having a wall and a main lumen, the main lumen configured to contain a main guidewire, the wall having an inner surface and an outer surface, the elongate member having at least one secondary lumen defined at least partially between the inner surface and outer surface of the wall, the at least one secondary lumen configured to releasably contain a secondary guidewire.

According to another example (“Example 16”), further to Example 15, the at least one secondary lumen is configured to sequentially release the secondary guidewire.

According to another example (“Example 17”), further to Example 15 or 16, the at least one secondary lumen is configured to release a first portion of the secondary guidewire when an implantable medical device is advanced along the main guidewire to a first longitudinal position proximate the first portion of the guidewire and configured to release a second portion of the guidewire when the implantable medical device is advanced along the main guidewire to a second longitudinal position proximate the second portion of the guidewire.

According to another example (“Example 18”), further to any one of Examples 15-17, an opening into the at least one secondary lumen is defined through the inner surface of the wall along a longitudinal length of the elongate member.

According to another example (“Example 19”), further to any one of Examples 15-17, an opening into the at least one secondary lumen is defined through the outer surface of the wall along a longitudinal length of the elongate member.

According to an example (“Example 20”), a method of delivering a medical device includes advancing an elongate member to a target site, the elongate member having a main lumen and a partition positioned in the main lumen of the elongate member, the partition defining at least one secondary lumen, the elongate member having a main guidewire positioned in the main lumen and a secondary guidewire positioned in the at least one secondary lumen; retracting the elongated member from the target while the partition, main guidewire, and secondary guidewire are retained at the target site; and advancing a main body device along the main guidewire, the main body device permanently disrupting the partition along a longitudinal length of the partition to release the secondary guidewire from the at least one secondary lumen of the partition.

The foregoing Examples are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 is an illustration of a wire management device in accordance with an embodiment;

FIG. 2 is an illustration of a wire management device in use in conjunction with an implantable device and delivery system in accordance with an embodiment;

FIG. 3 is an illustration of a wire management device and an implantable device in accordance with an embodiment;

FIG. 4 is an illustration of a cross section of a wire management device in accordance with an embodiment;

FIG. 5 is a side view of a wire management device with guidewire sheaths in accordance with an embodiment;

FIG. 6 is a schematic of an example method for preparing a partition of a wire management device in accordance with an embodiment;

FIGS. 7-18 show an example method of implementing a wire management device in use in conjunction with an implantable device and a delivery system in accordance with an embodiment;

FIG. 19 is an illustration of an embodiment of a wire management device with secondary lumens with access through an exterior surface of a wall in accordance with an embodiment; and

FIG. 20 is an illustration of an embodiment of a wire management device with secondary lumens with access through an interior surface of a wall in accordance with an embodiment.

### DETAILED DESCRIPTION

### Definitions and Terminology

This disclosure is not meant to be read in a restrictive manner. For example, the terminology used in the application should be read broadly in the context of the meaning those in the field would attribute such terminology.

Persons skilled in the art will readily appreciate that various aspects of the present disclosure can be realized by any number of methods and apparatus configured to perform the intended functions. Stated differently, other methods and apparatus can be incorporated herein to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

Certain relative terminology is used to indicate the relative position of components and features. For example, words such as “top”, “bottom”, “upper,” “lower,” “left,” “right,” “horizontal,” “vertical,” “upward,” and “downward” are used in a relational sense (e.g., how components or features are positioned relative to one another) and not in an absolute sense unless context dictates otherwise. Similarly, throughout this disclosure, where a process or method is shown or described, the method may be performed in any order or simultaneously, unless it is clear from the context that the method depends on certain actions being performed first.

With respect to terminology of inexactitude, the terms “about” and “approximately” may be used, in certain instances, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example.

As used herein, “couple” means join, connect, attach, adhere, affix, or bond, whether directly or indirectly, and whether permanently or temporarily.

### Description of Various Embodiments

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

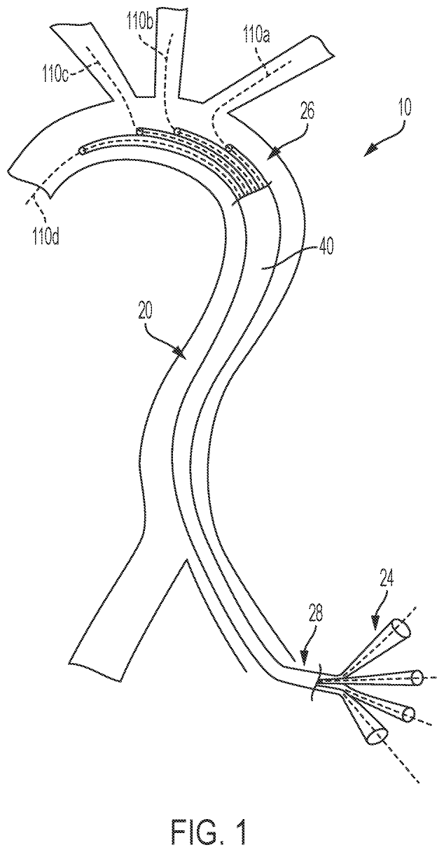

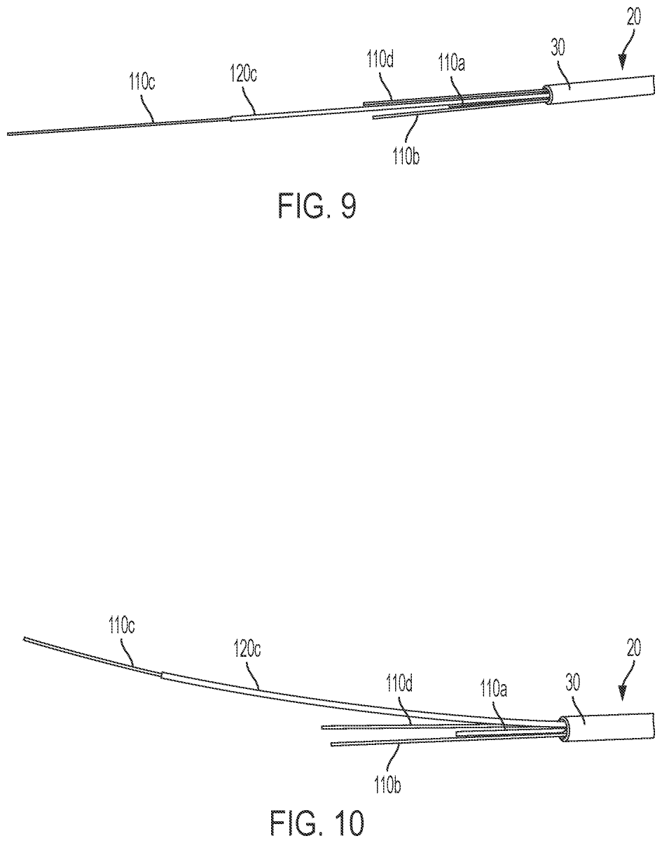

The device shown in FIG. 1 is provided as an example of the various features of the wire management device and, although the combination of those illustrated features is clearly within the scope of invention, that example and its illustration is not meant to suggest the inventive concepts provided herein are limited from fewer features, additional features, or alternative features to one or more of those features, for example as shown in FIGS. 8 and 9.

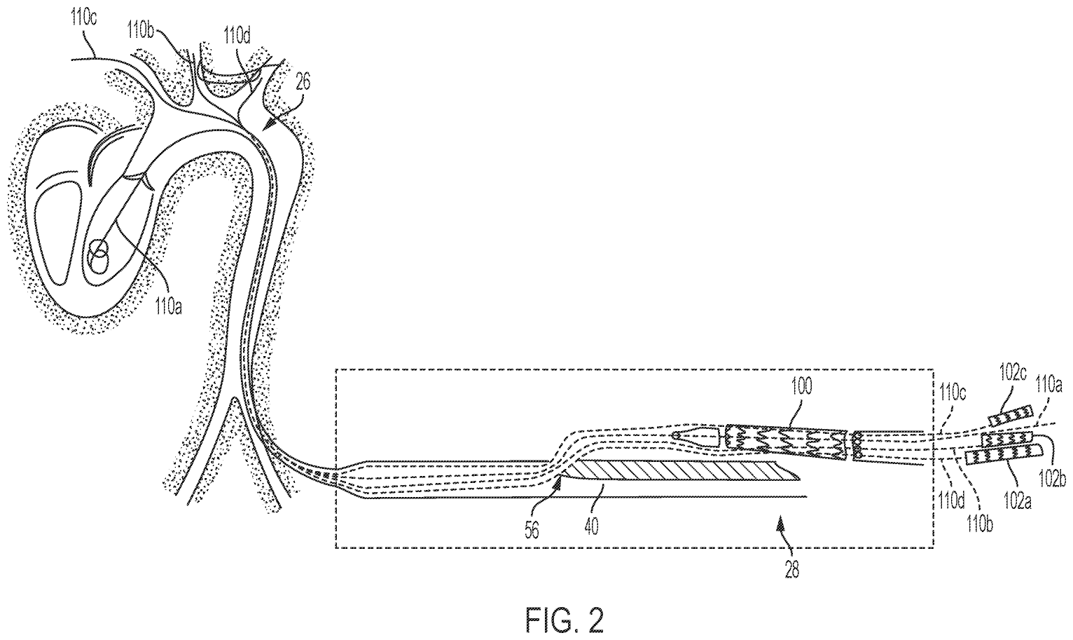

As shown in FIG. 1, a delivery device 10 including a wire management device 20 is illustrated. The wire management device 20 is operable to maintain and manage wires as the wires are advanced and positioned in the anatomy of a patient. For example, branched vasculature in a patient may be accessed for treatment and/or delivery of a device to a target site for addressing damaged or diseased tissue. In one example, the branched vasculature may include the aortic arch which is part of the aortic artery and includes branches into the brachiocephalic artery, the left common carotid artery and the left subclavian artery. Other exemplary branches may include renal arteries, mesenteric arteries, iliac arteries, and so forth. Furthermore, branches may be located in other arteries, veins, or other lumens such as in the lymphatic system, CSF passageways, and so forth. A device (e.g., implantable devices such as stents, grafts, and so forth), that may be delivered using the delivery device 10, may include a main body and device branches, device branches corresponding to some or all of the branches of the lumens to which the device is being delivered. The device branches may be coupled to, extend from, or may be positionable with the device. The device may be, for example, an implantable device 100 that may be delivered to the aortic arch. In certain instances, the implantable device 100 includes a first branch 102, a second branch 104, and a third branch 106 corresponding to each of the brachiocephalic, the left common carotid and the left subclavian arteries (e.g., see FIG. 2). In order to locate the implantable device 100 in the aortic arch and each of the branches 102 (e.g., 102a, 102b, 102c) in the corresponding arteries, a plurality of guidewires 110 (e.g., 110a, 110b, and 110c) are used to guide the respective elements to their target sites.

A wire management device 20 is provided to minimize interaction of each of the plurality of guidewires with each other and other components of the delivery device 10 in order to limit or prevent tangling, tying, or interference of the guidewires one with another and other components of the delivery device 10, which obstructs advancement of devices along the guidewires. The wire management device 20 maintains each of the guidewires in predetermined positions. The wire management device 20 is operable to release portions of the guidewires when a device is advanced along the longitudinal length of the wire management device 20, allowing the device and its branches to be advanced through the lumen of the patient. For example, the delivery system 10 may include a wire management device 20 that releasably contains a plurality of guidewires (e.g., a main guidewire 110a and a secondary guidewire 110b). The wire management device 20 may be configured to release a first portion of the at least one of the main guidewire 110a and secondary guidewire 110b when a device is advanced along the main guidewire 110a to a first longitudinal position proximate the first portion of the guidewires 110a, 110b and configured to release a second portion one of the guidewires 110a, 110b when the device is advanced along the main guidewire 110a to a second longitudinal position proximate the second portion of the guidewires 110a, 110b. Thus, the wire management device 20 progressively (described also as step-wise, inch-by-inch, or sequentially) releases the guidewires as a device is advanced with respect to the delivery device 10. This allows the guidewires to be appropriately positioned and to interact with the device (e.g., pass into an internal lumen of the device) in accordance with delivery of the device.

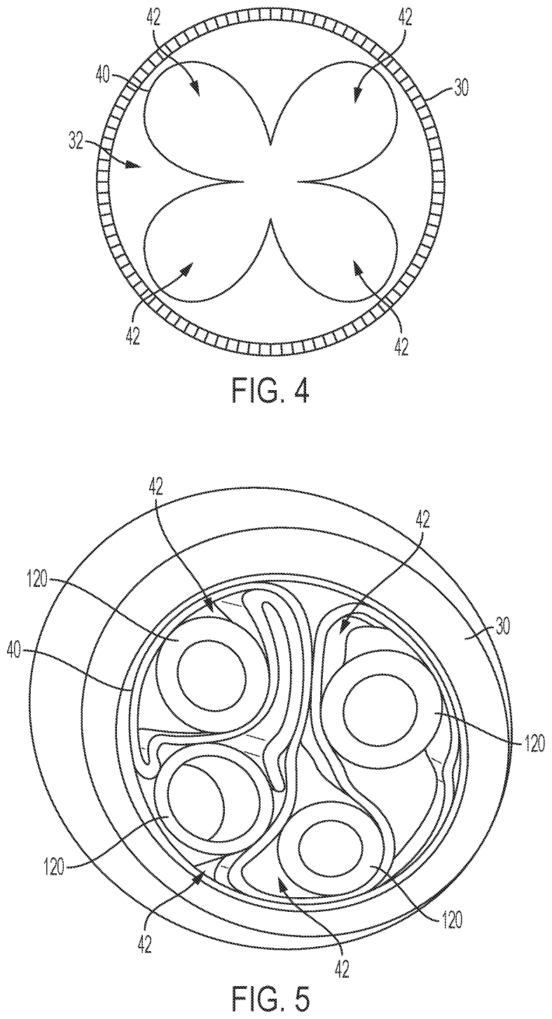

The wire management device 20 may include a plurality of lumens 22 or partial lumens within which the guidewires 110 may be removably contained (as shown in further detail, for example, in FIGS. 4 and 5). In some embodiments, the wire management device 20 includes ports 24 corresponding to each of the lumens 22 (as shown in FIG. 1). The ports 24 may be incorporated directly onto the wire management device 20 or may be positioned with the wire management device 20 such that each of the ports 24 provides access to a corresponding lumen of the wire management device 20.

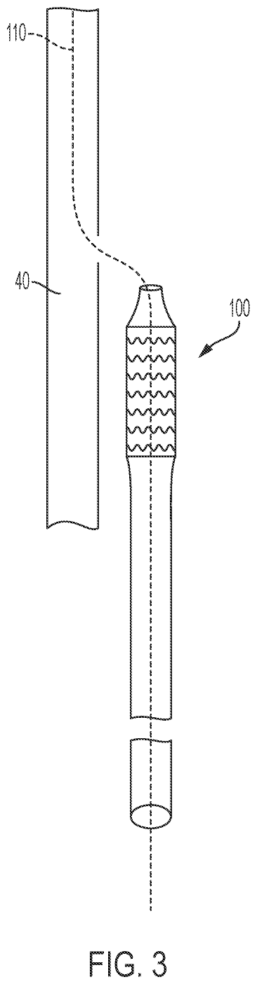

FIG. 2 illustrates an exemplary schematic of a delivery device 10 including a wire management device 20 for delivering an implantable device 100. The wire management device 20 includes a first end 26 and a second end 28. Guidewires 110 are positioned with the wire management device 20 such that the guidewires 110 extend from each of the first end 26 and the second end 28 of the wire management device 20 (see FIG. 1). Portions of the guidewires 110 may extend through various portions of the implantable device 100 (see FIG. 2). Other portions of the guidewires extend through the wire management device 20. The guidewires 110 may extend out from an end of the wire management device 20 into each of the corresponding portions of the lumens (e.g., into each of the corresponding branches of the vasculature, see FIG. 2). As the implantable device 100 is advanced along the guidewires 110, portions of the guidewires 110 are released from securement by the wire management device 20. The wire management device 20 may be used with one or more than one guidewire 110. FIG. 3, for example, is an illustration of a delivery device 10 implementing a single guidewire 110 in connection with a wire management device 20.

As various embodiments of the wire management device 20 is provided herein, a detailed discussion of each is provided individually. However, it is recognized that the features of any one of the following embodiments may be incorporated onto any of the other embodiments as appropriate.

Referring to FIG. 4, a first embodiment of a wire management device 20a includes a sheath 30 and a partition 40 (e.g., septum or divider). The sheath 30 forms a main lumen 32 along a longitudinal length of the sheath 30. The main lumen 32 has a sufficient diameter to accommodate the partition 40, guidewires 110, and guidewire sheaths 120. The sheath 30 is formed of a semi-flexible material that is sufficiently stiff to be advanced through anatomical lumens while being capable of bending in bends or curves in the anatomical lumens. For example, the sheath 30 may be formed of polyethylene, polytetrafluoroethylene, and so forth.

The partition 40 is operable to form a plurality of secondary lumens 42. Each of the secondary lumens 42 is operable to contain one of the guidewires 110. Because the partition 40 is positioned in the main lumen 32, the partition 40 divides the main lumen 32 to form at least one secondary lumen 42. FIGS. 4 and 5 are illustrated as having a partition 40 forming four secondary lumens 42. However, any number of secondary lumens 42 may be formed by the partition 40. For example, the partition may form one, two, three, four, five, or more secondary lumens 42.

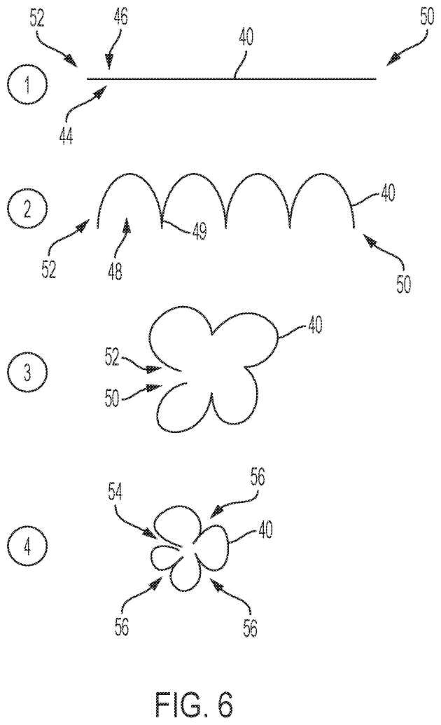

Referring to FIG. 6, the partition 40 may be formed of a sheet of material. The sheet of material may be folded or formed to define individual lumens (e.g., the secondary lumens 42). For example, in FIG. 6, the partition 40 is shown as having four secondary lumens 42. The partition 40 is folded such that each of the secondary lumens 42 is formed on a first side 44 of the sheet of material. More specifically, the partition 40 includes a first side 44 and a second side 46. Troughs 48 are formed on the first side 44 of the sheet of material, the troughs 48 being bounded by peaks 49. Each of the peaks 49 are then gathered together such that the troughs 48 each form the secondary lumens 42. Each of the peaks 49 can be coupled together (e.g., via an adhesive) or the peaks may be overlapping or positioned adjacent or abutting each other. However, an adhesive may be applied to the lateral ends 50, 52 of the sheet of material to secure the lateral ends 50, 52 to each other along the longitudinal length of the partition 40. The adhesive may be applied on the first or second side 44, 46 of the sheet of material, or both, as appropriate. The coupling of the lateral ends 50, 52 of the sheet of material forms a primary seam 54. The primary seam 54 is disruptable such that the primary seam 54 is released and sheet of material can be laid open. When the primary seam 54 is disrupted, the guidewires 110 can be removed from their corresponding secondary lumens 42.

In some embodiments, the primary seam 54 represents a position where a tear may be formed through the sheet of material (e.g., thinned sections of material, perforations, and so forth). Thus, the primary seam 54, when either adhesive is implemented and disrupted or the sheet of material is torn, is a permanent disruption. This allows the partition 40 to be removed from the delivery device 10 without having to implement additional steps such as tearing or cutting the partition 40 from around the guidewires 110 or a delivery handle (not shown). In those embodiments where adhesive is applied at each of the peaks 49, secondary seams 56 can be formed. However, the secondary seams 56 may be positioned such that when a secondary seam 56 is disrupted, adjacent secondary lumens 42 merge, but at least one secondary lumen 42 remains intact. It is noted that the partition 40 can be folded or formed to define multiple secondary lumens 42 without forming seals in the partition 40 between each secondary lumen 42.

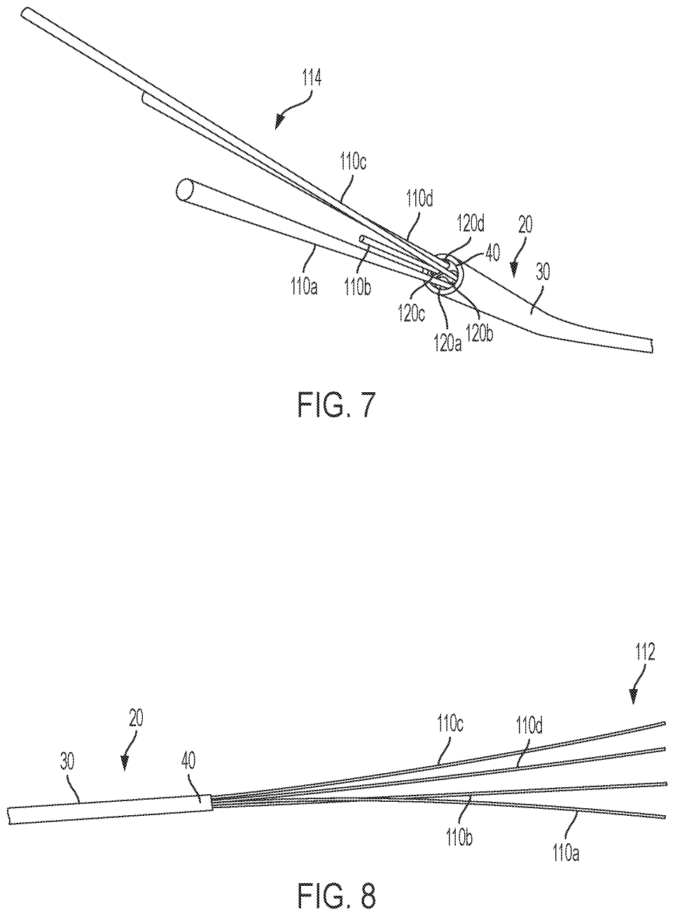

Referring now to FIGS. 7-18, an exemplary method of using the wire management device 20a of FIGS. 4 and 5 is illustrated. FIG. 7 shows the wire management device 20a, which includes the sheath 30 forming the main lumen 32 and the partition 40 positioned in the main lumen 32 and forming a plurality of secondary lumens 42 (for example, as shown in FIGS. 4 and 5). The wire management device 20a has a first guidewire sheath 120a with a first guidewire 110a, a second guidewire sheath 120b with a second guidewire 110b, a third guidewire sheath 120c with a third guidewire 110c, and a fourth guidewire sheath 120d with a fourth guidewire 110d. The wire management device 20a can be inserted into the patient (not shown), for example, into the femoral artery and advanced to the aortic arch. Each of the guidewires 110 can be appropriately positioned (e.g., in the brachiocephalic artery, the left common carotid artery, the left subclavian artery, and in the aortic arch). In some embodiments, some or all of the guidewire sheaths 120 are steerable. The first ends 112 of the guidewires 110 and/or guidewire sheaths 120 extend from the wire management device 20a (FIG. 8) such that the physician may appropriately manipulate the second ends 114 (FIGS. 9 and 10) of the guidewires 110 into the their respective positions and to install an implantable device (not shown) onto the first ends 112 such that it can be advanced from the first ends 112 toward the second ends 114 of the guidewires 110.

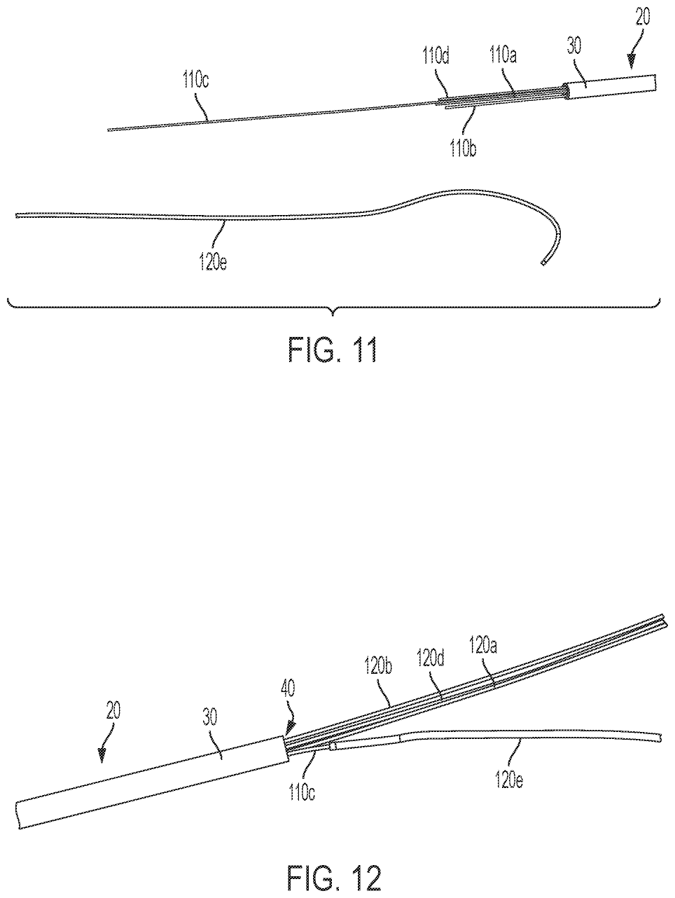

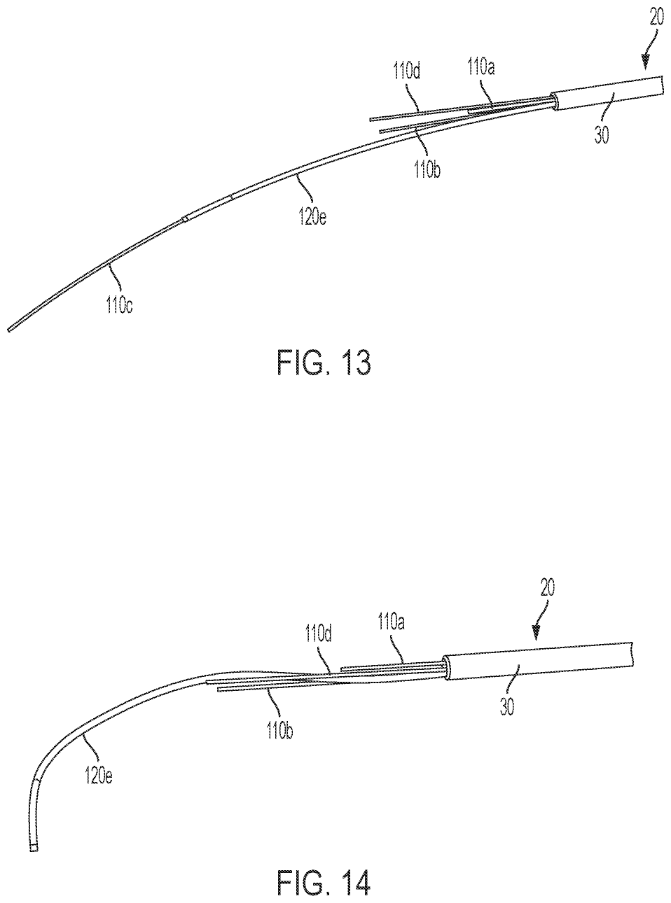

Referring to FIG. 11, in some embodiments, a steerable sheath 120e can be used in combination with the guidewires 110. As shown in FIG. 12, the steerable sheath 120e can be inserted around a guidewire 110 at the first end 112 and then advanced toward the second end 114 (FIG. 13). When the steerable sheath 120e is installed on the guidewire 110, the guidewire 110 can be steered into the appropriate position (FIG. 14).

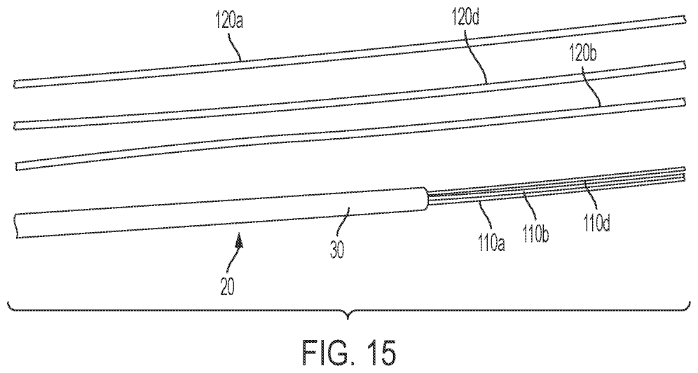

Once the guidewires 110 are appropriately positioned, the guidewire sheaths 120 can be removed from around the guidewires 110 be retracting the sheath from the second end 114 toward the first end 112 of the guidewires 110. The guidewire sheaths 120 are removed from the wire management device 20a (FIG. 15) when the guidewire sheaths 120 are removed from the guidewires 110.

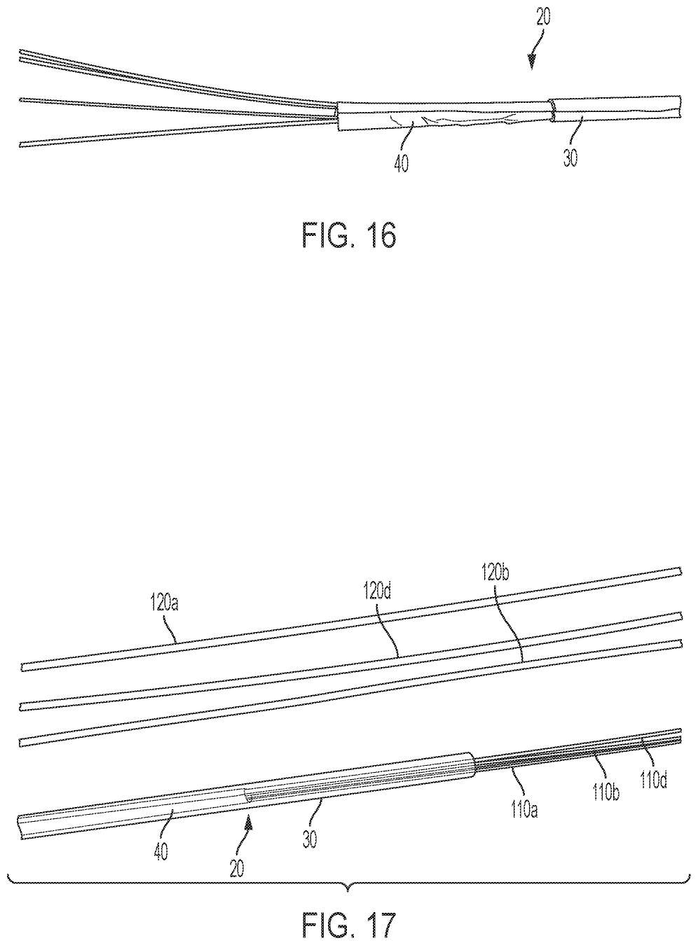

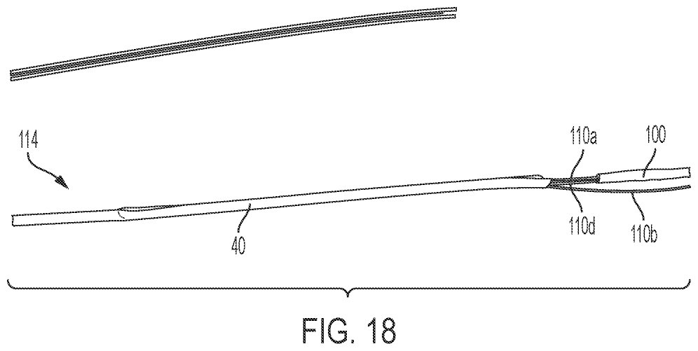

Referring to FIGS. 16 and 17, with all of the guidewires 110 appropriately positioned, the sheath 30 of the wire management device 20a can be removed from around the partition 40 and guidewires 110. Referring to FIG. 18, with the sheath 30 removed, the implantable device 100 can be inserted onto the first end 112 of the guidewires 110. In some embodiments, a single device is installed onto all of the guidewires 110. In these embodiments, the guidewires 110 may be positioned through various, separate features of the implantable device 100 (e.g., branches of a branched graft). In other embodiments, various, separate portions of an implantable device 100 may be installed on each guidewire 110 (e.g., branches can be installed onto single lumen graft). In these embodiments, the guidewires 110 may extend through side walls of the implantable device 100.

When the implantable device 100 is installed onto the guidewires 110, the implantable device 100 can be advanced along the guidewires 110. As the implantable device 100 is advanced toward the first end 112 of the guidewires, the implantable device 100 contacts the partition 40. As implantable device contacts the partition 40, the implantable device 100 disrupts the seam 56. As the seam 56 disrupts, the guidewires 110 are released from their respective secondary lumens 42 (FIG. 2). The seam 56 disrupts sequentially along the longitudinal length of the partition 40 as the implantable device 100 is advanced. Once the seam 56 has been disrupted along the full longitudinal length of partition 40, the guidewires 110 are no longer contained by the partition 40. However, the portions of the partition 40 where the seam 56 is not disrupted maintains the guidewires 110 in separate lumens (i.e., secondary lumens 42). In some embodiments, one or more of the guidewires 110 may not be positioned in the secondary lumens 42 of the partition 40, but instead were positioned in the main lumen 32, and accordingly were released from engagement when the sheath 30 was removed.

Once the partition 40 is removed, the implantable device 100 is located at or proximate the target site. Because the guidewires 110 were contained during delivery of the implantable device 100, the guidewires 110 were not tangled, crossed, or otherwise entangled which can cause improper seating of components at the target site, hindered advancement of the implantable device 100 along the guidewires 110, dislocation of the guidewires 110 from the target site (e.g., branches of a lumen), and so forth. The implantable device 100 is then deployed and the guidewires 110 are removed from the patient.

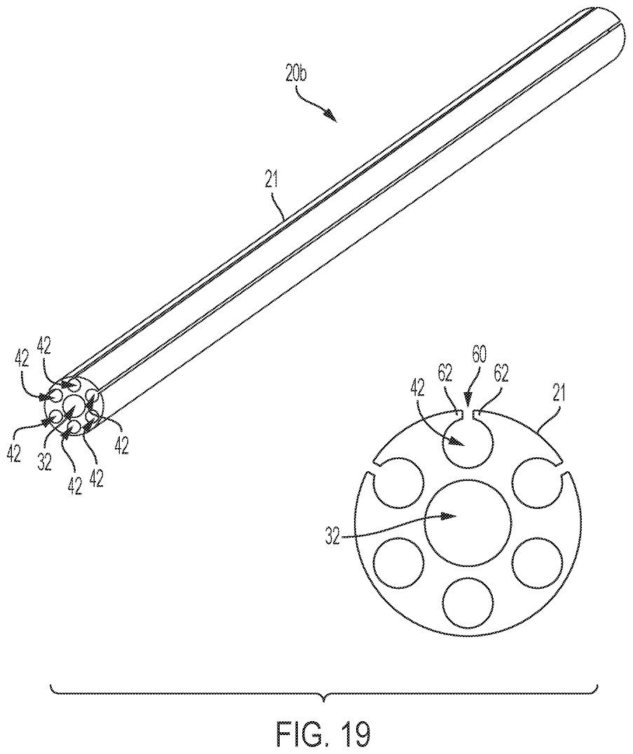

Referring now to FIG. 19, another embodiment of wire management device 20b is illustrated. In this embodiment, the wire management device 20b includes an elongate member 21 with a main lumen 32 and at least one secondary lumen 42. In the illustrated embodiment, the main lumen 32 is formed at the center and through the elongate member 21 along the longitudinal length of the wire management device 20b. The secondary lumens 42 are formed through the elongate member 21 along the longitudinal length of the wire management device 20b. The secondary lumens 42 do not overlap with the main lumen 32 with respect to a cross section across the longitudinal length. In some embodiments, the secondary lumens 42 can be positioned concentrically about the main lumen 32 such that the secondary lumens 42 and the main lumen 32 are separate and do not overlap. It is understood that the secondary lumens 42 may be positioned in any arrangement with respect to the main lumen 32 such that the secondary lumens 42 are separated from the main lumen 32. The separation can be provided via the body of the elongate member 21 (i.e., the lumens are formed within the body of the elongate member 21 separate from each other) or partition (not shown) is provided to separate the main lumen 32 and the secondary lumens 42.

At least some of the secondary lumens 42 may be accessed along the longitudinal length of the wire management device 20b. This can be provided by forming a slit 60 through a portion of the elongate member 21 connecting the exterior of the elongate member 21 with the secondary lumen 42. Stated otherwise, the secondary lumens 42 can be formed in the elongate member 21 with a partition 62 that is integral with the elongate body. The partition 62 provides resistance against movement from the secondary lumen 42 outward. The partition 62 is operable to flex or bend away from a neutral position when sufficient force is applied such that a guidewire 110 can be removed from the secondary lumen 42 through the slit 60 when sufficient force is applied against the partition. For example, when the secondary lumens 42 and the guidewires 110 have a generally circular profile, the secondary lumens 42 may be formed in the elongate member about a second lumen axis.

The secondary lumen 42 may be enclosed by the elongate member 21 and the integral partition 62 from about 189 to about 359 degrees, where the integral partition is the portion of the elongate member 21 that extends beyond 180 degrees such that the guidewires 110 are partially enclosed on a second side. The width of the slit 60 and accordingly the size of the integral partition 62 may be a function of the desired resistance required for disengaging the guidewire 110 from the wire management device 20. Furthermore, the material of the elongate member 21 can be varied to provide various resistances, either by incorporating a more flexible or compliant material or incorporating a stiff material. In some embodiments (not shown), the integral partition 62 may be a structural feature of the elongate member 21 that mates or corresponds with a feature of the guidewire 110 to retain the guidewire 110 in the secondary lumens 42 (e.g., a cap and ridge engagement, a keyed slot, and so forth). The guidewires 110 are retained by the wire management device 20b in either the main lumen 32 or the secondary lumens 42 until the guidewires are sequentially and selectively removed from the secondary lumens 42 as the guidewires 110 pass through the slit 60 and thus out of engagement at that position with the wire management device 20b.

The wire management device 20b may be dimensioned such that the implantable device 100 can be advanced through the patent lumen either adjacent to or surrounding the wire management device 20b. Some of the secondary lumens 42 of the wire management device 20b may be fully enclosed and therefore do not sequentially release the guidewires 110.

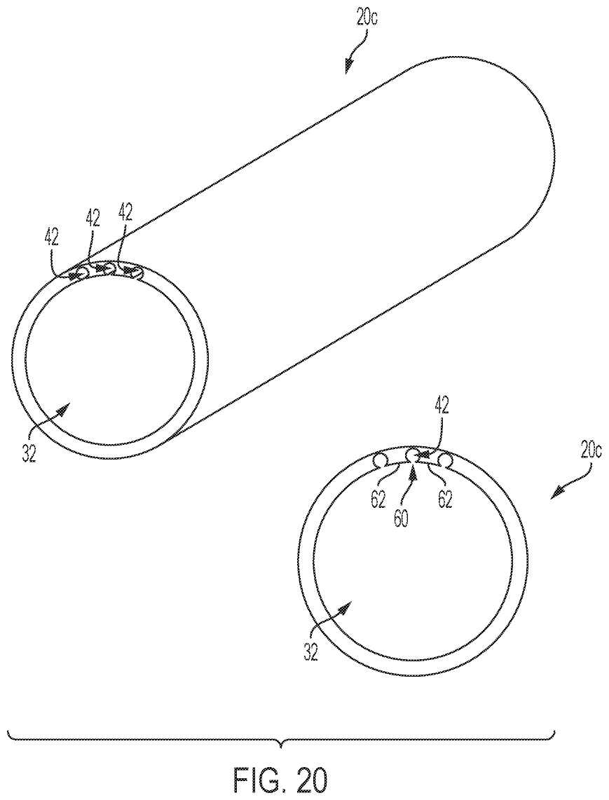

Referring now to FIG. 20, the wire management device 20c may be similar to the wire management device 20c of FIG. 19 in that the secondary lumens 42 are formed in the body of the elongate member. However, in contrast to the wire management device 20b of FIG. 19, the wire management device 20b of FIG. 20 includes secondary lumens 42 that are open to the main lumen 32. Thus, as the guidewires 110 are sequentially released from the secondary lumens 42, the guidewires 110 are released into the main lumen. In this embodiment, the wire management device 20c may include an outer support member (not shown) that radially supports the wire management device about the outer periphery (e.g., a film, sheet, material, wire mesh, and so forth) to reduce the risk of tearing through the wall of the elongate member 21. In this embodiment, the secondary lumens 42 may be positioned about the periphery of the wire management device 20c (not shown), or concentrated about a portion of the elongate member 21.

More specifically, the wire management device 20c includes an elongate member 21 with a main lumen 32 and at least one secondary lumen 42. In the illustrated embodiment, the main lumen 32 is formed at the center and through the elongate member 21 along the longitudinal length of the wire management device 20c. The secondary lumens 42 are formed through the elongate member 21 along the longitudinal length of the wire management device 20c. Access to the secondary lumens 42 is provided via the main lumen 32 along the longitudinal length of the elongate member 21. In some embodiments, the secondary lumens 42 can be positioned concentrically about the main lumen 32. It is understood that the secondary lumens 42 may be positioned in any arrangement with respect to the main lumen 32 such that the secondary lumens 42 are separated from the main lumen 32. The separation can be provided via the body of the elongate member 21 (i.e., the lumens are formed within the body of the elongate member 21 separate from each other) or partition (not shown) is provided to separate the main lumen 32 and the secondary lumens 42. In some embodiments, a small portion of the main lumen 32 and the secondary lumens 42 such that the lumens 32, 42 are open to each other due to their overlap. In some embodiments, the main lumen 32 and the secondary lumens 42 do not overlap with the main lumen 32 with respect to a cross section across the longitudinal length, although access is provided via a slit or gap.

At least some of the secondary lumens 42 may be accessed along the longitudinal length of the wire management device 20b. This can be provided by forming a slit 60 through a portion of the elongate member 21 connecting the main lumen 32 with the secondary lumen 42. Stated otherwise, the secondary lumens 42 can be formed in the elongate member 21 with a partition 62 that is integral with or extends from the elongate body to separate the lumens 32, 42. The partition 62 provides resistance against movement from the secondary lumen 42 into the main lumen 32. The partition 62 is operable to flex or bend away from a neutral position when sufficient force is applied such that a guidewire 110 can be removed from the secondary lumen 42 through the slit 60 when sufficient force is applied against the partition. For example, when the secondary lumens 42 and the guidewires 110 have a generally circular profile, the secondary lumens 42 may be formed in the elongate member about a second lumen axis. The secondary lumen 42 may be enclosed by the elongate member 21 and the integral partition 62 from about 189 to about 359 degrees, where the integral partition is the portion of the elongate member 21 that extends beyond 180 degrees such that the guidewires 110 are partially enclosed on a second side. The width of the slit 60 and accordingly the size of the integral partition 62 may be a function of the desired resistance required for disengaging the guidewire 110 from the wire management device 20. Furthermore, the material of the elongate member 21 can be varied to provide various resistances, either by incorporating a more flexible or compliant material or incorporating a stiff material.

In some embodiments (not shown), the integral partition 62 may be a structural feature of the elongate member 21 that mates or corresponds with a feature of the guidewire 110 to retain the guidewire 110 in the secondary lumens 42 (e.g., a cap and ridge engagement, a keyed slot, and so forth). The guidewires 110 are retained by the wire management device 20b in either the main lumen 32 or the secondary lumens 42 until the guidewires are sequentially and selectively removed from the secondary lumens 42 as the guidewires 110 pass through the slit 60 and thus out of engagement at that position with the wire management device 20b.

The wire management device 20b is dimensioned such that the implantable device 100 can be advanced through the patient lumen through the main lumen 32 of the wire management device 20b. Some of the secondary lumens 42 of the wire management device 20b may be fully enclosed and therefore do not sequentially release the guidewires 110.

The inventive scope of this application has been described above both generically and with regard to specific examples. It will be apparent to those skilled in the art that various modifications and variations can be made in the examples without departing from the scope of the disclosure. Likewise, the various components discussed in the examples discussed herein are combinable. Thus, it is intended that the examples cover the modifications and variations of the inventive scope.

1. A medical device delivery system comprising:
an elongate member having a main lumen configured to contain a main guidewire and at least one secondary lumen configured to releasably contain a secondary guidewire, the secondary lumen configured to release a first portion of the secondary guidewire when an implantable medical device is advanced along the main guidewire to a first longitudinal position proximate the first portion of the guidewire and configured to release a second portion of the guidewire when the implantable medical device is advanced along the main guidewire to a second longitudinal position proximate the second portion of the guidewire.

2. The medical device delivery system of claim 1, wherein the elongate member comprises a radially stiff material.

3. The medical device delivery system of claim 1, wherein the secondary lumen is formed in a wall of the elongate member.

4. The medical device delivery system of claim 1, wherein an opening into the secondary lumen is defined in the elongate member between the secondary lumen and the main lumen along a longitudinal length of the elongate member.

5. The medical device delivery system of claim 1, wherein an opening into the secondary lumen is defined through an outer radial surface of the elongate member along a longitudinal length of the elongate member.

6. The medical device delivery system of claim 1, further comprising a partition positioned in the main lumen of the elongate member, the partition defining the secondary lumen.

7. The medical device delivery system of claim 6, wherein the partition comprises a single sheet of material.

8. The medical device delivery system of claim 1, wherein the secondary lumen is operable to restrict the secondary guidewire from interweaving with the main guidewire along a length of the secondary wire contained by the secondary lumen.

9. A medical device delivery system comprising:
an elongate member having a main lumen configured to contain a main guidewire; and
a partition removably positioned within the elongate member, the partition forming at least one secondary lumen within the main lumen, the partition configured to contain a secondary guidewire along at least a portion of a longitudinal length of the elongate member, the partition configured to sequentially disrupt along the longitudinal length of the elongate member to free the secondary guidewire from the secondary lumen when the elongate member is removed from surrounding the partition and as an implantable medical device is advanced along the main guidewire, the partition including a disruptable seam.

10. The medical device delivery system of claim 9, wherein a portion of the partition is configured to release a portion of the secondary guidewire into a lumen of the medical device when the portion of the partition is disrupted.

11. The medical device delivery system of claim 9, wherein the partition subdivides the main lumen to include at least two secondary lumens.

12. The medical device delivery system of claim 9, wherein the partition comprises a sheet of material.

13. The medical device delivery system of claim 9, wherein the partition is folded to form the secondary lumen.

14. The medical device delivery system of claim 9, wherein the partition includes an adhesive for releasably coupling a first edge of the partition to a second edge of the partition.

15. A medical device delivery system comprising:
an elongate member having a wall and a main lumen, the main lumen configured to contain a main guidewire, the wall having an inner surface and an outer surface, the elongate member having at least one secondary lumen defined at least partially between the inner surface and outer surface of the wall, the at least one secondary lumen configured to releasably contain a secondary guidewire.

16. The medical device delivery system of claim 15, wherein the at least one secondary lumen is configured to sequentially release the secondary guidewire.

17. The medical device delivery system of claim 15, wherein the at least one secondary lumen is configured to release a first portion of the secondary guidewire when an implantable medical device is advanced along the main guidewire to a first longitudinal position proximate the first portion of the guidewire and configured to release a second portion of the guidewire when the implantable medical device is advanced along the main guidewire to a second longitudinal position proximate the second portion of the guidewire.

18. The medical device delivery system of claim 15, wherein an opening into the at least one secondary lumen is defined through the inner surface of the wall along a longitudinal length of the elongate member.

19. The medical device delivery system of claim 15, wherein an opening into the at least one secondary lumen is defined through the outer surface of the wall along a longitudinal length of the elongate member.

20. A method of delivering a medical device comprising:
advancing an elongate member to a target site, the elongate member having a main lumen and a partition positioned in the main lumen of the elongate member, the partition defining at least one secondary lumen, the elongate member having a main guidewire positioned in the main lumen and a secondary guidewire positioned in the at least one secondary lumen;
retracting the elongated member from the target while the partition, main guidewire, and secondary guidewire are retained at the target site; and
advancing a main body device along the main guidewire, the main body device permanently disrupting the partition along a longitudinal length of the partition to release the secondary guidewire from the at least one secondary lumen of the partition.
